(19) United States
(12) Patent Application Publication
(10) Pub. No.: US 2024/0313555 A1
(43) Pub. Date: Sep. 19, 2024

(54) APPARATUS AND ULTRASOUND IMAGING SYSTEM FOR HOLDING AND CHARGING WIRELESS ULTRASOUND PROBES

(71) Applicant: GE Precision Healthcare LLC, Wauwatosa, WI (US)

(72) Inventors: Tilman Noelle, Salzburg (AT); Lionel Wodecki, Maisons Alfort (FR); Sung Doo Lee, Seongnam (KR); Robert Andrew Meurer, Waukesha, WI (US)

(21) Appl. No.: 18/120,953

(22) Filed: Mar. 13, 2023

(51) Int. Cl. H02J 7/00; A61B 8/00

(52) U.S. Cl. CPC H02J 7/0044; A61B 8/4472

(57) ABSTRACT

Systems and methods are provided for holding and charging wireless ultrasound probes. A probe holder may be configured to securely engage, at least, a probe charger that is configured to engage a wireless ultrasound probe used in an ultrasound imaging system. The probe holder may include one or more securing elements configured to secure in place at least the probe charger once engaged with the probe holder. The probe holder may be configured to engage a corresponding part of the ultrasound imaging system, with the probe holder secured in place, once engaged with the ultrasound imaging system, based on a securing mechanism. At least one securing element may be configured to secure in place, at least in part, both of the probe charger and the wireless ultrasound probe. The probe holder may include one or more second securing elements configured to secure the probe holder based on the securing mechanism.

### FIELD

Aspects of the present disclosure relate to ultrasound imaging solutions. More specifically, certain embodiments of the present disclosure relate to systems and methods for holding and charging wireless ultrasound probes.

### BACKGROUND

Ultrasound imaging may be used for medical imaging based examinations, such as in imaging organs and soft tissues in a human body. In this regard, the manner by which images are generated during ultrasound imaging depends on the particular technique. For example, ultrasound imaging uses real time, non-invasive high frequency sound waves to produce ultrasound images, typically of organs, tissues, objects (e.g., fetus) inside the human body. Images produced or generated during ultrasound imaging may be two-dimensional (2D), three-dimensional (3D), and/or four-dimensional (4D) images (essentially real-time/continuous 3D images). During ultrasound imaging, imaging datasets (including, e.g., volumetric imaging datasets during 3D/4D imaging) are acquired and used in generating and rendering corresponding images (e.g., via a display) in real-time.

In some instances, operation of certain components of ultrasound imaging systems, such as the ultrasound imaging probes, may pose certain challenges and/or may have some limitations, particularly with respect to ease of use, and conventional and traditional approaches may not sufficiently address or overcome these challenges.

Further limitations and disadvantages of conventional and traditional approaches will become apparent to one of skill in the art, through comparison of such systems with some aspects of the present disclosure, as set forth in the remainder of the present application with reference to the drawings.

### BRIEF SUMMARY

An apparatus and an ultrasound imaging system are provided for holding and charging wireless ultrasound probes, substantially as shown in and/or described in connection with at least one of the figures, as set forth more completely in the claims.

These and other advantages, aspects and novel features of the present disclosure, as well as details of one or more illustrated example embodiments thereof, will be more fully understood from the following description and drawings.

### BRIEF DESCRIPTION OF THE DRAWINGS

FIG. 1 is a block diagram illustrating an example ultrasound imaging system.

FIG. 2 illustrates an example ultrasound imaging system that may be configured to incorporate and support use of wireless ultrasound probes and associated charging and holding arrangements, in accordance with an example embodiment based on the present disclosure.

FIG. 3 illustrates an example ultrasound imaging system having a wireless ultrasound probe and associated charging and holding arrangement, in accordance with an example embodiment based on the present disclosure.

FIG. 4 illustrates use of two different positions for wireless ultrasound probes and associated charging and holding arrangements, in accordance with an example embodiment based on the present disclosure.

FIG. 5 illustrates a top side of an example probe holder that may be used in wireless ultrasound probe-based charging and holding arrangements, in accordance with an example embodiment based on the present disclosure.

FIG. 6 illustrates features of a bottom side of an example probe holder used during installation of the probe holder, in accordance with an example embodiment based on the present disclosure.

FIG. 7 illustrates a probe charger installed on a probe holder in an example wireless ultrasound probe-based charging and holding arrangement, in accordance with an example embodiment based on the present disclosure.

FIG. 8 illustrates exemplary steps for installing an example wireless ultrasound probe-based charging and holding arrangement, in accordance with an example embodiment based on the present disclosure.

FIG. 9 illustrates various holding features in an example wireless ultrasound probe-based charging and holding arrangement, in accordance with an example embodiment based on the present disclosure.

### DETAILED DESCRIPTION

Certain embodiments of the present disclosure may be found in systems and methods for holding and charging wireless ultrasound probes. Various embodiments have the technical effect of optimizing operation of ultrasound imaging systems, particularly systems incorporating and utilizing wireless ultrasound probes, by use of probe holders that are configured for facilitating secure holding, and charging, of such wireless ultrasound probes. An example probe holder based on the present disclosure may be configured to securely engage, at least, a probe charger, which in turn may be configured to engage a wireless ultrasound probe used in an ultrasound imaging system. The probe charger may incorporate securing element(s) configured to secure in place at least the probe charger once engaged with the probe holder. The probe holder may be further configured to engage a corresponding part of the ultrasound imaging system with a securing mechanism to securely hold the probe holder in place. The probe holder may comprise or be made from a transparent material. At least one securing element may be configured to secure in place, at least in part, both of the probe charger and the wireless ultrasound probe. The at least one securing element may comprise, for example, a hook-like structure extending from an edge of the probe holder and configured to engage one or both of a side part and a top part of one or both of the probe charger and the wireless ultrasound probe. The hook-like structure may comprise, for example, a double-curve structure that may comprise a first part for engaging the probe charger and a second part for engaging the wireless ultrasound probe. At least one other securing element may comprise a protrusion-like structure extending from a top-side of the probe holder. The protrusion-like structure of the at least one other securing element may be configured to engage a corresponding part on a bottom-side of the probe charger, or a corresponding feature on the bottom-side of the probe charger. The probe holder comprise one or more additional securing elements configured to engage with the ultrasound imaging system to secure the probe holder in place on the ultrasound imaging system. In various embodiments, the securing mechanism may be a magnetic force-based mechanism, where the securing mechanism may comprise at least one structure configured to house a magnet or a steel (or other ferrous material based) bracket. The at least one structure may be at a location opposite a location of at least one corresponding structure on the corresponding part of the ultrasound imaging system, with the corresponding structure being configured to house a magnet, or to house a steel (or other ferrous material based) bracket when the at least one structure houses a magnet. The corresponding part of the ultrasound imaging system may comprise one or more recesses configured to receive the probe holder, with the probe holder configured to reside or fit within each of the one or more recesses. One or both of the probe holder and the corresponding part of the ultrasound imaging system are configured to enable placement of the probe holder at a right-side position or a left-side position with respect to a user of the ultrasound imaging system. One or both of the probe holder and the probe charger may be configured such that when engaged to one another there is space between the probe holder the probe charger to allow for air flow. The probe charger may comprise one or more ports, each configured to receive a connector for providing power from the ultrasound imaging system, such as via a charging cable. The probe holder may comprise one or more structures configured to secure in place the charging cable once plugged into the port(s).

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” as used in the context of ultrasound imaging is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI Angio, and in some cases also MM, CM, TVD where the “image” and/or “plane” includes a single beam or multiple beams. In addition, as used herein, the phrase “pixel” also includes embodiments where the data is represented by a “voxel.” Thus, both the terms “pixel” and “voxel” may be used interchangeably throughout this document.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC, or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. In addition, forming of images may be performed using channel combinations that may originate from more than one transmit event (e.g., synthetic aperture techniques).

In various embodiments, processing to form images is performed in software, firmware, hardware, or a combination thereof. The processing may include use of beamforming.

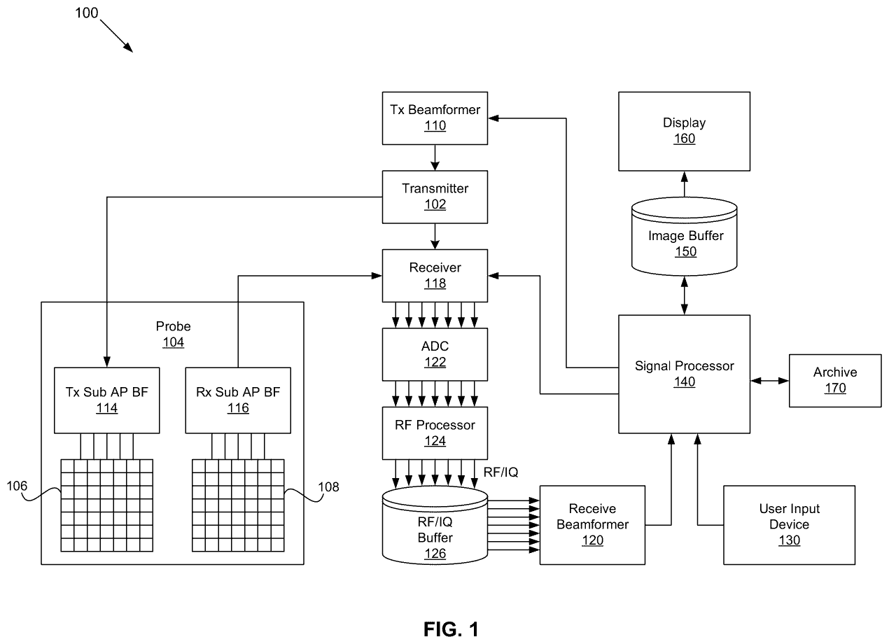

FIG. 1 is a block diagram illustrating an example ultrasound imaging system. Shown in FIG. 1 is an ultrasound imaging system 100. The ultrasound imaging system 100 may be configured for providing ultrasound imaging, and as such may comprise suitable circuitry, interfaces, logic, and/or code for performing and/or supporting ultrasound imaging related functions. As shown in FIG. 1, the ultrasound imaging system 100 comprises, for example, a transmitter 102, an ultrasound probe 104, a transmit beamformer 110, a receiver 118, a receive beamformer 120, a RF processor 124, a RF/IQ buffer 126, a user input module 130, a signal processor 140, an image buffer 150, a display system 160, an archive 170.

The transmitter 102 may comprise suitable circuitry, interfaces, logic, and/or code that may be operable to drive an ultrasound probe 104. For example, the ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiments, the ultrasound probe 104 may be operable to acquire ultrasound image data covering at least a substantial portion of an anatomy, such as the heart, a blood vessel, or any suitable anatomical structure. The ultrasound probe 104 is not limited to embodiment illustrated in FIG. 1, however, and as such in other embodiments the ultrasound probe 104 may incorporate other arrangements of elements, such as a 1D array, and/or may utilized other suitable types of transducers, such as micromachined ultrasound transducer (MUT) elements, capacitive micromachined ultrasonic transducer (cMUT) elements, and the like.

The transmit beamformer 110 may comprise suitable circuitry, interfaces, logic, and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable circuitry, interfaces, logic, and/or code that may be operable to receive the signals from the receive sub-aperture beamformer 116. The analog signals may be communicated to one or more of the plurality of A/D converters 122.

The plurality of A/D converters 122 may comprise suitable circuitry, interfaces, logic, and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable circuitry, interfaces, logic, and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable circuitry, interfaces, logic, and/or code that may be operable to provide temporary storage of the RF or I/Q signal data, which is generated by the RF processor 124.

The receive beamformer 120 may comprise suitable circuitry, interfaces, logic, and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 140. In accordance with some embodiments, the receiver 118, the plurality of A/D converters 122, the RF processor 124, and the beamformer 120 may be integrated into a single beamformer, which may be digital. In various embodiments, the ultrasound imaging system 100 comprises a plurality of receive beamformers 120.

The user input device 130 may be utilized to input patient data, scan parameters, settings, select protocols and/or templates, and the like. In an example embodiment, the user input device 130 may be operable to configure, manage and/or control operation of one or more components and/or modules in the ultrasound imaging system 100. In this regard, the user input device 130 may be operable to configure, manage and/or control operation of the transmitter 102. the ultrasound probe 104, the transmit beamformer 110, the receiver 118, the receive beamformer 120, the RF processor 124, the RF/IQ buffer 126, the user input device 130, the signal processor 140, the image buffer 150, the display system 160, and/or archive 170. For example, the user input device 130 may include button(s), rotary encoder(s), a touchscreen, motion tracking, voice recognition, a mouse device, keyboard, camera and/or any other device capable of receiving user directive(s). In certain embodiments, one or more of the user input devices 130 may be integrated into other components, such as the display system 160 or the ultrasound probe 104, for example.

As an example, user input device 130 may include a touchscreen display. As another example, user input device 130 may include an accelerometer, gyroscope, and/or magnetometer attached to and/or integrated with the probe 104 to provide gesture motion recognition of the probe 104, such as to identify one or more probe compressions against a patient body, a pre-defined probe movement or tilt operation, or the like. In some instances, the user input device 130 may include, additionally or alternatively, image analysis processing to identify probe gestures by analyzing acquired image data. In accordance with the present disclosure, the user input and functions related thereto may be configured to support use of new data storage scheme, as described in this disclosure. For example, the user input device 130 may be configured to support receiving user input directed at triggering and managing (where needed) application of separation process, as described herein, and/or to provide or set parameters used in performing such process. Similarly, the user input device 130 may be configured to support receiving user input directed at triggering and managing (where needed) application of the recovery process, as described herein, and/or to provide or set parameters used in performing such process.

The signal processor 140 may comprise suitable circuitry, interfaces, logic, and/or code that may be operable to process ultrasound scan data (i.e., summed IQ signal) for generating ultrasound images for presentation on a display system 160. The signal processor 140 is operable to perform one or more processing operations according to a plurality of selectable ultrasound modalities on the acquired ultrasound scan data. In an example embodiment, the signal processor 140 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 160 and/or may be stored at the archive 170.

The archive 170 may be a local archive, a Picture Archiving and Communication System (PACS), or any suitable device for storing images and related information, or may be coupled to such device or system for facilitating the storage and/or achieving of the imaging related data. In an example implementation, the archive 170 is further coupled to a remote system such as a radiology department information system, hospital information system, and/or to an internal or external network (not shown) to allow operators at different locations to supply commands and parameters and/or gain access to the image data.

The signal processor 140 may be one or more central processing units, microprocessors, microcontrollers, and/or the like. The signal processor 140 may be an integrated component, or may be distributed across various locations, for example. The signal processor 140 may be configured for receiving input information from the user input device 130 and/or the archive 170, generating an output displayable by the display system 160, and manipulating the output in response to input information from the user input device 130, among other things. The signal processor 140 may be capable of executing any of the method(s) and/or set(s) of instructions discussed herein in accordance with the various embodiments, for example.

The ultrasound imaging system 100 may be operable to continuously acquire ultrasound scan data at a frame rate that is suitable for the imaging situation in question. Typical frame rates range from 10-220 but may be lower or higher. The acquired ultrasound scan data may be displayed on the display system 160 at a display-rate that can be the same as the frame rate, or slower or faster. The image buffer 150 is included for storing processed frames of acquired ultrasound scan data that are not scheduled to be displayed immediately. Preferably, the image buffer 150 is of sufficient capacity to store at least several minutes' worth of frames of ultrasound scan data. The frames of ultrasound scan data are stored in a manner to facilitate retrieval thereof according to its order or time of acquisition. The image buffer 150 may be embodied as any known data storage medium.

In some implementations, the signal processor 140 may be configured to perform or otherwise control at least some of the functions performed thereby based on a user instruction via the user input device 130. As an example, a user may provide a voice command, probe gesture, button depression, or the like to issue a particular instruction, such as to initiate and/or control various aspects of the color Doppler improvement function(s), and/or to provide or otherwise specify various parameters or settings relating thereto, as described in this disclosure.

In operation, the ultrasound imaging system 100 may be used in generating ultrasonic images, including two-dimensional (2D), three-dimensional (3D), and/or four-dimensional (4D) images. In this regard, the ultrasound imaging system 100 may be operable to continuously acquire ultrasound scan data at a particular frame rate, which may be suitable for the imaging situation in question. For example, frame rates may range from 30-70 but may be lower or higher. The acquired ultrasound scan data may be displayed on the display system 160 at a display-rate that can be the same as the frame rate, or slower or faster. An image buffer 150 is included for storing processed frames of acquired ultrasound scan data not scheduled to be displayed immediately. Preferably, the image buffer 150 is of sufficient capacity to store at least several seconds' worth of frames of ultrasound scan data. The frames of ultrasound scan data are stored in a manner to facilitate retrieval thereof according to its order or time of acquisition. The image buffer 150 may be embodied as any known data storage medium.

In some instances, the ultrasound imaging system 100 may be configured to support grayscale and color based operations. For example, the signal processor 140 may be operable to perform grayscale B-mode processing and/or color processing. The grayscale B-mode processing may comprise processing B-mode RF signal data or IQ data pairs. For example, the grayscale B-mode processing may enable forming an envelope of the beam-summed receive signal by computing the quantity (I²+Q²)^{1/2}. The envelope can undergo additional B-mode processing, such as logarithmic compression to form the display data. The display data may be converted to X-Y format for video display. The scan-converted frames can be mapped to grayscale for display. The B-mode frames that are provided to the image buffer 150 and/or the display system 160. The color processing may comprise processing color based RF signal data or IQ data pairs to form frames to overlay on B-mode frames that are provided to the image buffer 150 and/or the display system 160. The grayscale and/or color processing may be adaptively adjusted based on user input—e.g., a selection from the user input device 130, for example, for enhance of grayscale and/or color of particular area.

In some instances, ultrasound imaging may include generation and/or display of volumetric ultrasound images—that is where objects (e.g., organs, tissues, etc.) are displayed three-dimensional 3D. In this regard, with 3D (and similarly 4D) imaging, volumetric ultrasound datasets may be acquired, comprising voxels that correspond to the imaged objects. This may be done, e.g., by transmitting the sound waves at different angles rather than simply transmitting them in one direction (e.g., straight down), and then capture their reflections back. The returning echoes (of transmissions at different angles) are then captured, and processed (e.g., via the signal processor 140) to generate the corresponding volumetric datasets, which may in turn be used in creating and/or displaying volume (e.g. 3D) images, such as via the display 150. This may entail use of particular handling techniques to provide the desired 3D perception. For example, volume rendering techniques may be used in displaying projections (e.g., 3D projections) of the volumetric (e.g., 3D) datasets. In this regard, rendering a 3D projection of a 3D dataset may comprise setting or defining a perception angle in space relative to the object being displayed, and then defining or computing necessary information (e.g., opacity and color) for every voxel in the dataset. This may be done, for example, using suitable transfer functions for defining RGBA (red, green, blue, and alpha) value for every voxel.

In some instances, use of wireless devices or components in ultrasound imaging systems may be desirable, such as for enhanced portability and ease of operation. For example, in some implementations wireless ultrasound probes may be used. In this regard, a wireless ultrasound probe may be configured to operate as described herein (e.g., with respect to the probe 104), but additionally may be configured to utilized wireless connectivity (e.g., Bluetooth, Wi-Fi, or the like) to communicate with the remaining part of the ultrasound imaging system, such as in conjunction with the transmitting of ultrasound signals and capturing/receiving echo signals corresponding thereto, and/or in conjunction with control of operation of the probe during ultrasound imaging operations. However, use of such wireless ultrasound probes may pose some challenges and/or may have some limitations. In particular, such wireless ultrasound probes may need to be (re-)charged as they are not connected (e.g., via cords or the like) to the ultrasound imaging system, and thus cannot be driven directly by the system. Also, there is a need to ensure that these probes are housed in secure manner. Solutions in accordance with the present disclosure may allow for enhanced use of such wireless ultrasound probes, particularly compared to conventional solutions (if any existed), such as with respect to aspects like the housing (holding) of such probes and charging thereof.

In various example implementations based on the present disclosure, ultrasound imaging systems (e.g., the ultrasound imaging system 100) may be configured to hold and charge wireless ultrasound probes used in these systems, such as by incorporating holding and charging arrangements, components, and/or features that are particularly designed and/or configured to ensure that the wireless ultrasound probes are held securely, are charged (e.g., when engaged with a probe charger), and are readily accessible for use by system operators. Example implementations and additional details related thereto are described in more detail below.

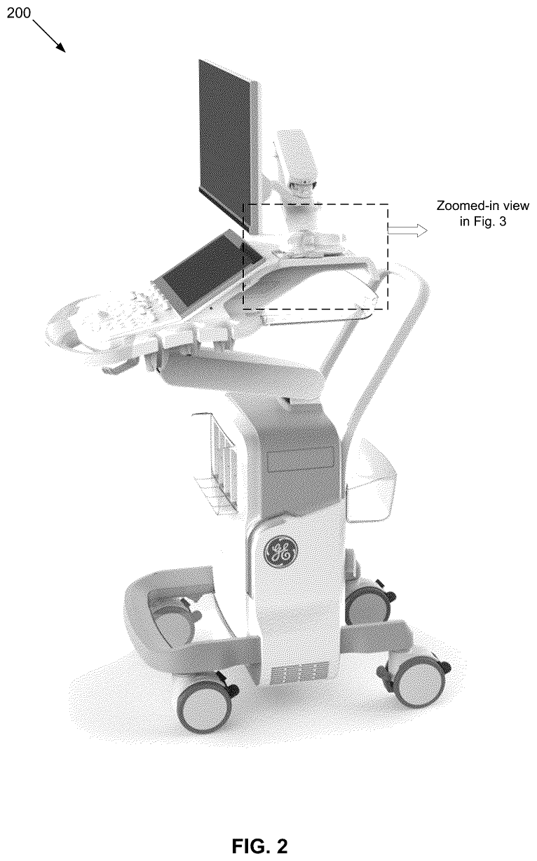

FIG. 2 illustrates an example ultrasound imaging system that may be configured to incorporate and support use of wireless ultrasound probes and associated charging and holding arrangements, in accordance with an example embodiment based on the present disclosure. Referring to FIG. 2, an ultrasound imaging system 200 is shown.

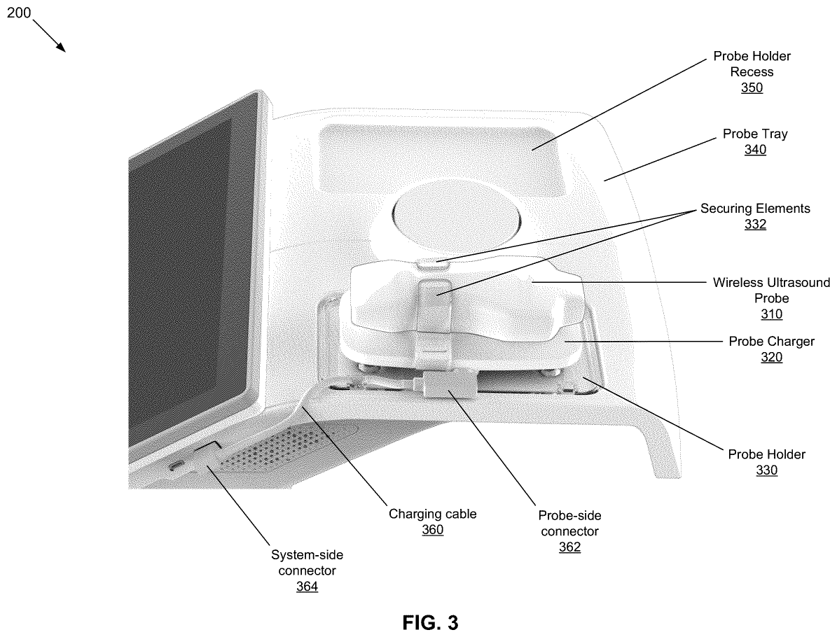

The ultrasound imaging system 200 may be similar to, and represents an example embodiment of the ultrasound imaging system 100 of FIG. 1. However, the ultrasound imaging system 200 is configured to incorporate and support use of wireless ultrasound probes and associated charging and holding arrangements as described herein. In this regard, the ultrasound imaging system 200 may incorporate components added and/or modified to facilitate using wireless ultrasound probes—that is, probes that are not connected to the system using cords, cables, or the like. In accordance with the present disclosure, supporting use of wireless ultrasound probes may entail adding or modifying components of the ultrasound imaging system 200 to enable housing, holding, and charging such wireless ultrasound probes, and to do so in an optimal manner. In particular, the components used in holding and charging the wireless ultrasound probes may be designed and installed in an easily accessible position for storing and charging the wireless ultrasound probes—e.g., where probes may be ready for use and in reach for the user. Example implementations are shown and described in more detail with respect to FIGS. 3-9.

As illustrated, the ultrasound imaging system 200 is a portable system—that is, it may be readily movable by system operators, such as by designing or implementing various components of the system so they may fit on single movable cabinet (or similar structure) that incorporates means for allowing it to be moved (e.g., such as wheels as shown in FIG. 2). The disclosure is not limited to portable imaging systems, however, and implementations based on the disclosure may be used in other types of systems, which may not be portable.

FIG. 3 illustrates an example ultrasound imaging system having a wireless ultrasound probe and associated charging and holding arrangement, in accordance with an example embodiment based on the present disclosure. Referring to FIG. 3, the ultrasound imaging system 200 of FIG. 2, and in particular a portion thereof that may house and support use of wireless ultrasound probe-based holding and charging arrangements, is shown.

In particular, in the example implementation illustrated in FIG. 3, the ultrasound imaging system 200 may comprise a probe tray 340, which is a flat (or tray-like) structure that is particularly designed or configured to receive and support components configured for holding and charging wireless ultrasound probes. The probe tray 340 may be incorporated into the system 200, or may be an add-on part that may be added to existing systems, such as behind a display or monitor of the system, as shown in FIG. 3. The probe tray 340 may incorporate one or more features or structures that are specifically configured to receive the wireless ultrasound probe holding and charging components. For example, the probe tray 340 may incorporate one or more probe holder recesses 350, each being specifically designed to receive a corresponding probe holder 330, which in turn may be configured to receive and hold a probe charger 320 and a wireless ultrasound probe 310.

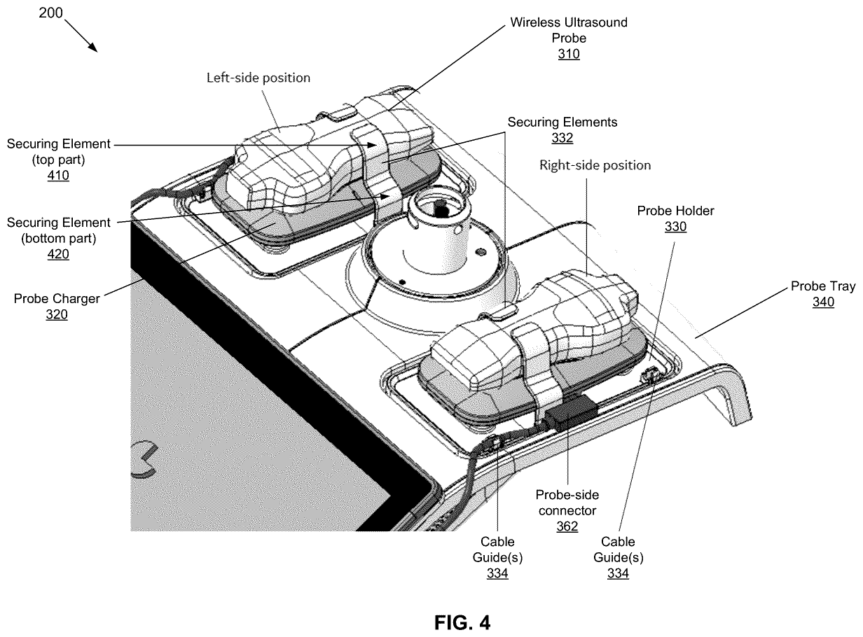

In some instances, the wireless ultrasound probe and associated charging and holding arrangement may be placed or deployed on either side of the tray—that is, the right-side or left-side of the probe tray 340, within either one of the two probe holder recesses 350. This is illustrated in and described in more detail with respect to FIG. 4.

The wireless ultrasound probe 310 may be any suitable wireless ultrasound probe that is configured for use in conjunction with medical (e.g., ultrasound) imaging operation as described herein. In this regard, the wireless ultrasound probe 310 may represent a wireless based embodiment of the ultrasound probe 104 as described with respect to FIG. 1. The probe charger 320 may be adaptively designed (e.g., shaped to fit or otherwise configured to engage) for engaging one or more particular types of probes. For example, the probe charger 320 may be physically designed or built to optimally match or fit the shape and size of particular probe(s). Similarly, the probe holder 330 may be adaptively designed (e.g., shaped to fit or otherwise configured to engage) for engaging one or more particular types of probe chargers and/or probes. For example, the probe holder 330 may be physically designed or built, and/or may incorporate features (e.g., structures), that optimally match or fit the shape and size of particular probe holder(s) and/or probe(s).

The probe charger 320 may be configured to charge wireless ultrasound probes inserted into, or otherwise coupled to (e.g., in contact with) the probe charger 320. The charging may be by use of induction, for example, but the disclosure is not limited to such approach, and any suitable approach for charging the probes, once engaged with the probe charger may be used. The probe charger 320 may be configured to receive from suitable sources, such as the ultrasound imaging system 200 itself, power required for operation of the probe charger 320, such as when charging any probe inserted or otherwise coupled to the probe charger 320. For example, as illustrated in FIG. 3, once inserted and secured in place, a charging cable 360 may be used to connect the probe charger 320 to the ultrasound imaging system 200 to provide power to the probe charger 320. In this regard, the probe charger 320 may incorporate one or more ports, each supporting a particular type of connectors (e.g., USB based connectors or the like), with a probe-side connector 362 of the charging cable 360 being plugged into one such port. Similarly, a system-side connector 364 of the charging cable 360 may be connected to the ultrasound imaging system 200, such as via a suitable port into a corresponding port (e.g., an external USB port) therein.

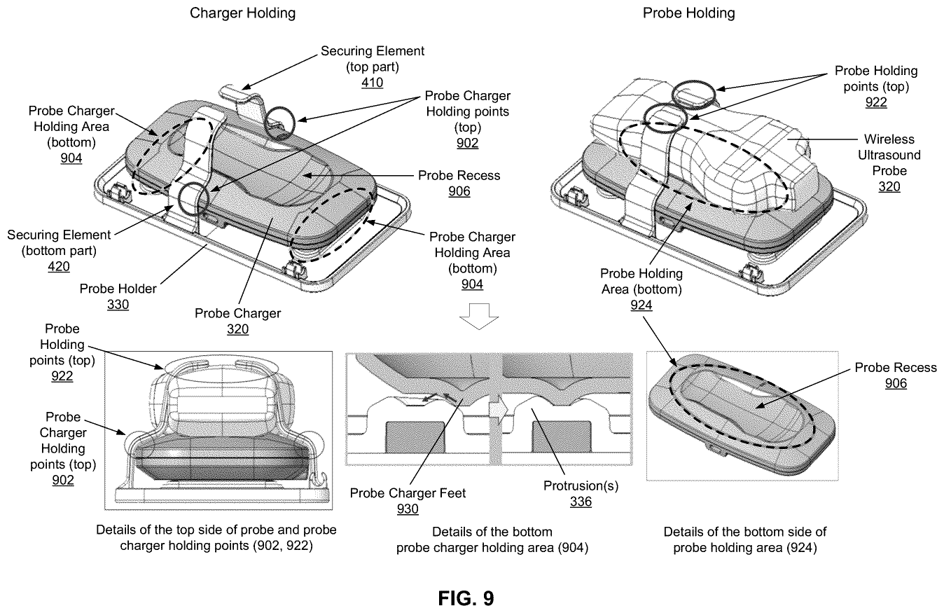

The probe holder 330 may be configured to facilitate or support, alone or in conjunction with other components, securing one or both of probe charger 320 and the wireless ultrasound probe 310 once inserted. For example, as illustrated in FIG. 3, the probe holder 330 may incorporate securing elements 332 which may be configured to facilitate or support, alone or in conjunction with other features or structures in the probe holder 330 and/or in other components, securing one or both of probe charger 320 and the wireless ultrasound probe 310 once inserted. As illustrated in FIG. 3 and some of the other figures, the securing elements 332 may incorporate a hook-like structure, comprising, e.g., a double-curve like design, with a first part of the double-curve, such as bottom part(s) 420 of the securing elements 332, adaptively designed or built to securely engage the probe charger 320, and a second part of the double-curve, such as top part(s) 410 of the securing elements 332, adaptively designed or built to securely engage the wireless ultrasound probe 310. This is illustrated more clearly in FIG. 9.

The disclosure is not limited to such design, however, and in some instances other approaches may be used, including, e.g., use of separate securing structures and/or features for securing the probe charger 320 and the wireless ultrasound probe 310, respectively. Also, the probe holder 330 may be configured to secure only the probe charger 320, with the wireless ultrasound probe 310 being secured by other components, such as the probe charger 320 itself. In such implementations, the probe charger 320 may incorporate separate securing structures and/or features for securing the wireless ultrasound probes.

In some implementations, the probe holder 330, including all components and features thereof (e.g., the securing elements 332), may be constructed to optimize usability of the ultrasound imaging system by system operator(s). For example, the probe holder 330, including all components and features thereof (e.g., the securing elements 332), may be constructed such that its presence in the system may not be a distraction to the system operator(s). The probe holder 330, including all components and features thereof (e.g., the securing elements 332) may be constructed such that it may be transparent—that is, comprising or being made from suitable transparent material, such as clear plastic or the like. Such transparent design may be preferable, such as to avoid distracting the user since the transparency of the probe holder 330 would make it easier to see and thus grab the wireless ultrasound probe.

The various components of the wireless ultrasound probe holding and charging arrangement illustrated in FIG. 3, and various features and functions thereof, are illustrated and described in more detail with respect to FIGS. 4-9.

FIG. 4 illustrates use of two different positions for wireless ultrasound probes and associated charging and holding arrangements, in accordance with an example embodiment based on the present disclosure. Referring to FIG. 4, the combination of the wireless ultrasound probe 310, the probe charger 320, and the probe holder 330 installed into the probe tray 340 is shown.

In particular, illustrated in FIG. 4 is the combination of the wireless ultrasound probe 310, the probe charger 320, and the probe holder 330 installed in each of the two probe holder recesses 350 of the probe tray 340, thus demonstrating the installation of the combination in each of the left-side position and the right-side position of the probe tray 340 in relation to the operator of the ultrasound imaging system 200, which presumably would be facing the screen(s) as shown in the ultrasound imaging system 200 of FIG. 2. Also illustrated in FIG. 4 is that the probe holder 330 may comprise cable guide(s) 334 guiding the connection of the charging cables 360 to the probe charger 320. In this regard, the ultrasound imaging system 200 may support connecting the charging cables 360 in each of the left-side position and the right-side position, such as by incorporating a suitable port (e.g., an external USB port) on either side of the probe tray 340 where the system-side connector 364 may be plugged.

Further, as shown in FIG. 4, the combination of the wireless ultrasound probe 310, the probe charger 320, and the probe holder 330 as a whole may be rotated such that the port of the probe charger 320, where the probe-side connector 362 is plugged, may be to the outside in each of the left-side position and the right-side position. Alternatively, in another implementation, the probe charger 320 and the probe holder 330 may be configured to support installation in the left-side position and the right-side position without requiring rotation of the components. For example, the probe holder 330 may incorporate cable guides 334 on both sides, and the probe charger may also (optionally) incorporate connector ports on both side.

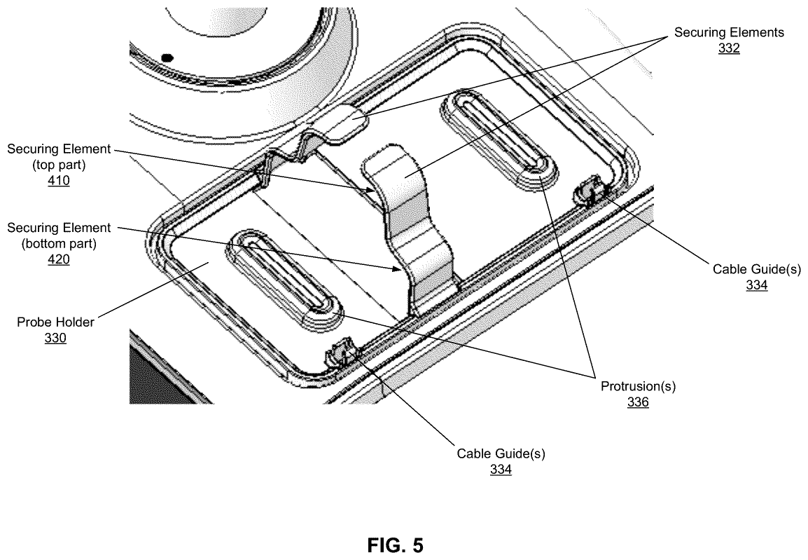

FIG. 5 illustrates a top side of an example probe holder that may be used in wireless ultrasound probe-based charging and holding arrangements, in accordance with an example embodiment based on the present disclosure. Referring to FIG. 5, the probe holder 330, illustrating specifically the top-side of the probe holder 330 and features thereof is shown.

In this regard, the top-side of the probe holder 330 may incorporate, in addition to the securing elements 332 described above, features configured for supporting other functions, such as, e.g., engaging the probe charger 320, engaging the probe holder recesses 350 when the probe holder 330 is installed therein, securing the charging cables, etc. For example, as illustrated in FIG. 5, the top-side of the probe holder 330 incorporates cable guide(s) 334 and protrusion(s) 336. In this regard, the cable guide(s) 334 may be configured to engage and secure (in place) the charging cables when plugged into the probe charger 320 and the imaging system. The cable guide(s) 332 may incorporate, e.g., hook or snap-in-place based structure.

The protrusion(s) 336 may be configured to engage the probe charger 320 (or parts thereof) when inserted into the probe holder 330. For example, the protrusions 336 may be configured to engage corresponding features (e.g., feet or the like) of the probe charger 320. The use of the protrusions 336 may enable or further enhance securing the probe charger 320 (in place) once inserted into the probe holder 330. The protrusions 336 may also provide or support additional functions, such as cooling. In this regard, the use of the protrusions 336, particularly in conjunction with corresponding features in the probe charger 320 (e.g., feet thereof), may ensure that there would be space between the probe holder 330 and the probe charger 320, which in turn allows for air flow thus providing passive cooling of the probe charger 320 during charging operations, which may obviate the need to use dedicated active cooling components or devices, such as cooling fans or the like.

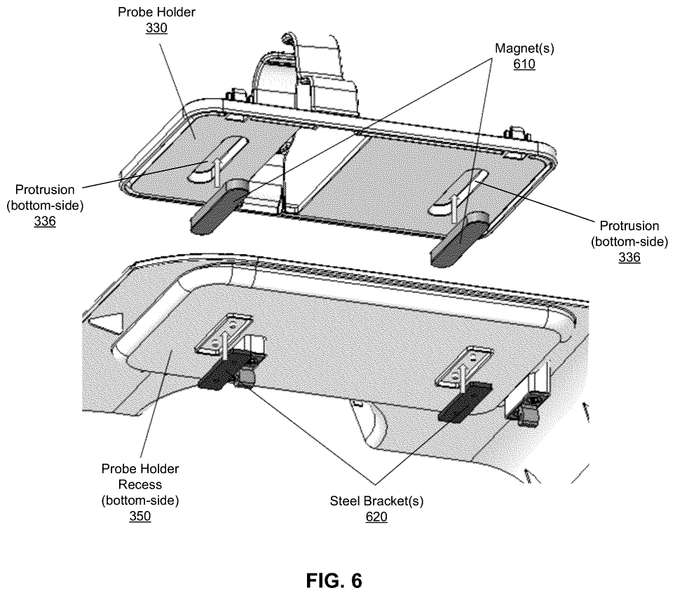

In some instances, the protrusions 336 may be disposed (e.g., formed) on the opposite side—that is, the bottom-side of the probe holder 330—recess areas, which may be used for coupling or otherwise securing the probe holder onto the probe tray 340 (within the probe holder recesses 350). This is shown in and described in more detail with respect to FIG. 6.

FIG. 6 illustrates features of a bottom side of an example probe holder used during installation of the probe holder, in accordance with an example embodiment based on the present disclosure. Referring to FIG. 6, the probe holder 330, illustrating specifically the bottom-side of the probe holder 330 and various features thereof is shown.

In this regard, the bottom-side of the probe holder 330 may incorporate features configured for supporting functions associated with use of the probe holder 330, such as engaging the probe tray 340, or particularly the probe holder recesses 350 that house probe holders such as the probe holder 330, for example. Such features may be specifically configured to facilitate securing in place the probe holder 330 in accordance with particular securing mechanism. For example, the probe holder 330 may be secured to the probe tray 340 using magnetic forces. In this regard, utilization of magnetic forces based securing mechanism may entail use of a combination of magnets, or magnets and steel pieces (or any suitable ferrous material based pieces), incorporated on corresponding parts of the probe holder 330 and the probe tray 340.

For example, as illustrated in FIG. 6, the probe holder 330 may incorporate magnet(s) 610, which would engage corresponding steel brackets 620 that may be incorporated into probe tray 340 to provide the necessary magnetic forces to secure the probe holder 330 in place. However, it should be understood that the disclosure is not limited to this example implementation, and as such in other implementations, other similarly suitable arrangements may be used—e.g., with magnets incorporated into the probe tray 340 and steel brackets incorporated into the probe holder 330 instead, or with suitable magnets (e.g., providing magnetic attractive pull) incorporated into both of the probe tray 340 and the probe holder 330.

With reference to the implementation illustrated in FIG. 6, the magnets 610 may be incorporated into the bottom-side of the probe holder 330, for example. In this regard, as noted above, the bottom-side of the probe holder 330, and particularly features therein, may be configured to facilitate or support the securing mechanism and components required for use thereof, such as when utilizing the magnets 610 for facilitating use of magnetic forces to secure in place the probe holder 330. For example, as illustrated in FIG. 6, the protrusions 336 on the top-side of the probe holder 330 may form recesses on the bottom-side of the probe holder 330, into which the magnets 610 may be inserted or otherwise placed. Further, the probe tray 340 may incorporate, in the areas opposite of the recesses created by the protrusions 336 within the probe holder recesses 350, features for incorporating the steel brackets 620, as illustrated in FIG. 6. In this regard, in the example implementation shown in FIG. 6, the steel brackets 620 are inserted into bottom-side of the probe tray 340/probe holder recess 350 (and thus requiring receiving features therein). It should be understood, however, that the disclosure is not limited to use of magnet based implementations, nor to use of the specific magnetic based implementation illustrated in FIG. 6.

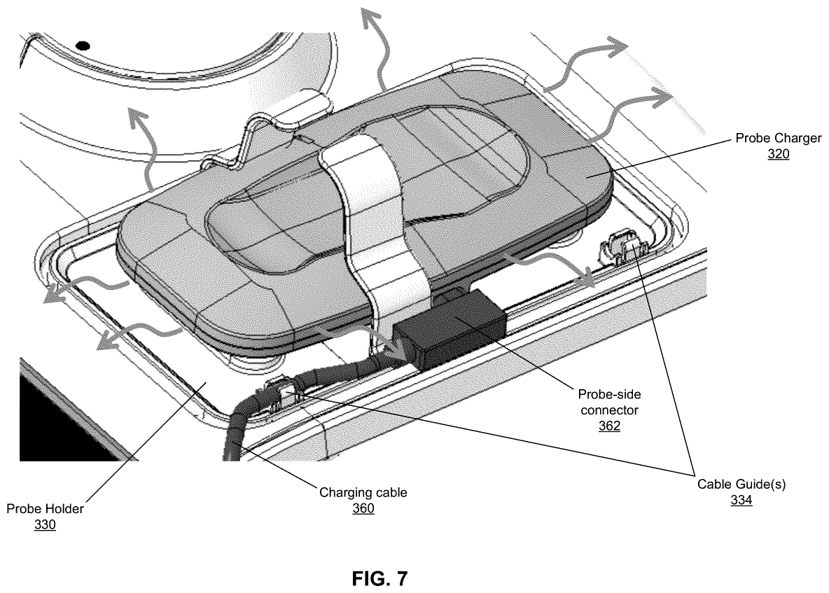

FIG. 7 illustrates a probe charger installed on a probe holder in an example wireless ultrasound probe-based charging and holding arrangement, in accordance with an example embodiment based on the present disclosure. Referring to FIG. 7, a probe charger 320 inserted into a probe holder 330 it is shown.

In this regard, as described herein, once the probe holder 330 is fixed or otherwise coupled (e.g., using magnetic forces) to the probe tray 340, specifically within one of the probe holder recesses 350, the probe charger 320 may be inserted or otherwise coupled to the probe holder 330. In particular, the probe charger 320 may be inserted such that the securing elements 332 of the probe holder 330 secure in place the probe charger 320. The holding of the probe charger 320 within or by the probe holder 330 is illustrated in and described in more detail with respect to FIG. 9. Once inserted and secured in place, the cable probe charger 360 may be connected to the probe charger 320 to provide the power required when, e.g., charging any probe inserted or otherwise coupled to the probe charger 320. The probe charger 320 may incorporate one or more ports, each supporting a particular type of connector (e.g., USB based connector or the like), with the probe-side connector 362 of the charging cable 360 plugged into a suitable port. Once plugged, the charging cable 360 may be secured in place, such as using the cable guide(s) 334 of the probe holder 330, as shown in FIG. 7.

As noted above, the top-side of the probe holder 330 may incorporate the protrusions 336, which may be configured to engage corresponding features (e.g., feet or the like) in the probe charger 320 to further enhance securing in place the probe charger 320 once inserted into the probe holder 330. Further, as noted above, the combination of the protrusions 336 and the corresponding features (e.g., feet) in the probe charger 320 may also ensure that there would be space between the probe holder 330 and the probe charger 320, to allow for air flow as shown in FIG. 7 (the arrows flowing from bottom of the probe charger 320), thus providing passive cooling of the probe charger 320 during charging operations.

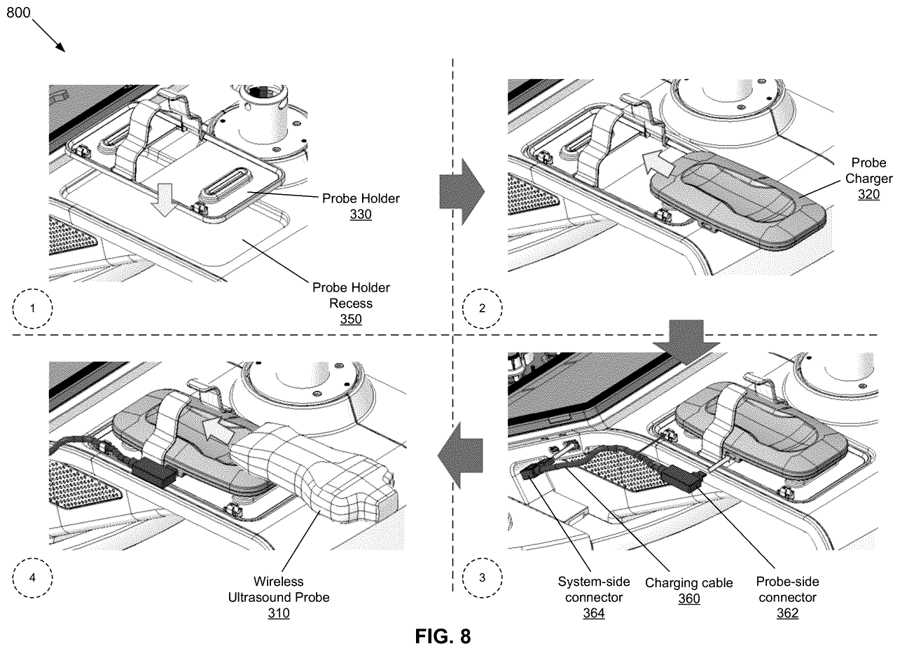

FIG. 8 illustrates exemplary steps for installing an example wireless ultrasound probe-based charging and holding arrangement, in accordance with an example embodiment based on the present disclosure. Referring to FIG. 8, a process 800 for assembling and installing a wireless ultrasound probe and associated charging and holding arrangement is shown.

In a first step (step 1), the probe holder 330 may be installed onto the probe 340 tray, particularly within one of the probe holder recesses 350. In this regard, the probe holder may be secured to the probe tray using particular securing mechanism, such as based on magnetic forces, e.g., as illustrated in and described with respect to FIG. 6.

In a second step (step 2), the probe charger 320 may be installed—e.g., slid into, or otherwise engaged to the probe holder 330. In this regard, the feet of the probe charger 320 may be fitted onto the protrusions 336 of the probe holder 330. Furthermore, the securing elements 332 of the probe holder 330, or a portion thereof (e.g., a lower/first curve of the double-curve structure of the securing elements 332—that is, the bottom part(s) 420) may engage outer top edges of the probe charger 320 to secure the probe charger 320 in place on the probe holder 330 between the securing elements 332.

In a third step (step 3), the charging cable 360 may be connected, e.g., with probe-side connector 362 plugged into the probe charger 320, and the system-side connector 364 plugged into a corresponding port in the system (e.g., an external USB port). In this regard, the charging cable 360 may also be inserted into cable guide(s) 334 of probe holder 330.

In a fourth step (step 4), the wireless ultrasound probe 310 may be installed—e.g., slid into, or otherwise engaged to the probe holder 330, engaging the probe charger 320 already installed therein. Furthermore, the securing elements 332 of the probe holder 330, or a portion thereof (e.g., an upper/second curve of the double-curve structure of the securing elements 332—that is, the top part(s) 410) may engage the wireless ultrasound probe charger 310 to secure the probe 310 in place on the probe charger 320 and probe holder 330 between the securing elements 332.

FIG. 9 illustrates various holding features in an example wireless ultrasound probe-based charging and holding arrangement, in accordance with an example embodiment based on the present disclosure. Referring to FIG. 9, a combination of the wireless ultrasound probe 310, the probe charger 320, and the probe holder 330 illustrates the various holding features used therein to secure in place these different components.

In this regard, as described herein once the probe holder 330 is fixed or otherwise coupled into the probe tray 340 of the ultrasound imaging system 200, specifically within one of the probe holder recesses 350, the probe charger 320 may be inserted or otherwise coupled to the probe holder 330, and then the wireless ultrasound probe 310 may be inserted or otherwise coupled to the probe charger 320. Various holding features, incorporated into one or more of these devices or components, may be used to secure in place these devices or components.

For example, as illustrated in FIG. 9, with respect to the holding and/or securing of the probe charger 320, the combination of the protrusions 336 of the probe holder 330, alone or in combination with corresponding features in the probe charger 320 (e.g., feet 930), may provide probe charger holding area(s) 904 at the bottom-side of the probe charger 320, as shown in the more detailed (zoomed in) picture. Further, the securing elements 332 of the probe holder 330 may provide top-side holding points 902 for the probe charger 320. In this regard, in instances where the securing element 332 utilizes a double-curve hook-like shaped structure, the bottom part(s) 420 of the securing elements 332 may provide the top-side holding points 902 of the probe charger 320.

With respect to the probe holding/securing, the probe charger 320 may incorporate features (e.g., a recessed area 906) on its top-side, which may provide, alone or in combination with corresponding features in the wireless ultrasound probe 310 (e.g., matching shape thereof), a bottom-side probe holding area 924 for the wireless ultrasound probe 310. Further, the securing elements 332 of the probe holder 330 may provide top-side holding points 922 for the probe holder 330. In this regard, in instances where the securing element 332 utilizes a double-curve hook-like shaped structure, the top part(s) 410 of the securing elements 332 may provide the top-side holding points 922 of the wireless ultrasound probe 310. Accordingly, the shape of the securing elements 332 may be specifically set or adjusted to ensuring providing the top-side holding of the probe charger 320 and the wireless ultrasound probe 310.

An example apparatus, based on the present disclosure, for holding and charging a wireless ultrasound probe used in an ultrasound imaging system comprises a probe holder configured to securely engage, at least, a probe charger that is configured to engage the wireless ultrasound probe. The probe holder comprises one or more securing elements configured to secure in place at least the probe charger once engaged with the probe holder. The probe holder is configured to engage a corresponding part of the ultrasound imaging system. The probe holder is configured for being secured in place, once engaged with the ultrasound imaging system, based on a securing mechanism.

In an example embodiment, the probe holder comprises or is made from a transparent material.

In an example embodiment, the one or more securing elements comprise at least one element that is configured to secure in place, at least in part, both of the probe charger and the wireless ultrasound probe.

In an example embodiment, the one or more securing elements comprise at least one element that comprises a hook-like structure extending from an edge of the probe holder and configured for engaging one or both of a side part and a top part of one or both of the probe charger and the wireless ultrasound probe.

In an example embodiment, the hook-like structure comprises a double-curve structure comprising a first part for engaging the probe charger and a second part for engaging the wireless ultrasound probe.

In an example embodiment, the one or more securing elements comprise at least one element that comprises a protrusion-like structure extending from a top-side of the probe holder when engaged with the corresponding part, the protrusion-like structure configured to engage a bottom-side of the probe charger or a corresponding feature on the bottom-side of the probe charger.

In an example embodiment, the probe holder comprise one or more second securing elements configured to secure in place the probe holder, once engaged with the ultrasound imaging system, based on the securing mechanism.

In an example embodiment, the securing mechanism comprises magnetic force based mechanism, and wherein the one or more second securing elements comprise at least one structure configured to house a magnet or a steel or iron-based bracket.

In an example embodiment, the at least one structure is on a bottom-side of the probe holder.

In an example embodiment, the at least one structure is at a location that is opposite a location of at least one corresponding structure on the corresponding part of the ultrasound imaging system, and where the corresponding structure is configured to house a magnet, or to house a steel or iron-based bracket when the at least one structure houses a magnet.

In an example embodiment, the corresponding part of the ultrasound imaging system comprises one or more recesses configured to receive the probe holder, and wherein the probe holder is configured to reside or fit with each of the one or more recesses.

In an example embodiment, one or both of the probe holder and the corresponding part of the ultrasound imaging system are configured to enable placement of the probe holder at a right-side position or a left-side position with respect to a user of the ultrasound imaging system.

An example apparatus, based on the present disclosure, for holding and charging a wireless ultrasound probe used in an ultrasound imaging system, comprises a probe charger configured to engage the wireless ultrasound probe, and a probe holder configured to engage at least the probe charger. The probe charger is configured to charge the wireless ultrasound probe when engaged therewith. The probe holder comprises one or more securing elements, and wherein at least one securing element is configured to secure in place at least the probe charger once engaged with the probe holder. The probe holder is configured for being secured in place once engaged with the ultrasound imaging system.

In an example embodiment, one or both of the probe holder and the probe charger are configured such that when engaged to one another there is space between the probe holder the probe charger to allow for air flow.

In an example embodiment, the one or more securing elements comprise at least one element that comprises a hook-like structure extending from an edge of the probe holder and configured for engaging one or both of a side part and a top part of one or both of the probe charger and the wireless ultrasound probe.

In an example embodiment, the hook-like structure comprises a double-curve structure comprising a first part for engaging the probe charger and a second part for engaging the wireless ultrasound probe.

In an example embodiment, the probe charger comprises one or more structures configured for securing in place, at least in part, the wireless ultrasound probe once engaged therewith.

In an example embodiment, the probe charger comprises a port configured to receive a connector for providing power from the ultrasound imaging system via a charging cable.

In an example embodiment, the probe holder comprises one or more structures configured to secure in place the charging cable once plugged into the port.

An example ultrasound imaging system, based on the present disclosure, comprises a wireless ultrasound probe, a probe charger configured to engage the wireless ultrasound probe, and a probe holder configured to engage at least the probe charger. The probe charger is configured to charge the wireless ultrasound probe when engaged therewith. The probe holder comprises one or more securing elements, and wherein at least one securing element is configured to secure in place at least the probe charger once engaged with the probe holder. The wherein the probe holder is configured for being secured in place once engaged with the ultrasound imaging system.

As utilized herein the terms “circuits” and “circuitry” refer to physical electronic components (e.g., hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y, and z.” As utilized herein, the terms “block” and “module” refer to functions than can be performed by one or more circuits. As utilized herein, the term “exemplary” means serving as a non-limiting example, instance, or illustration. As utilized herein, the terms “for example” and “e.g.,” set off lists of one or more non-limiting examples, instances, or illustrations. As utilized herein, circuitry is “operable” to perform a function whenever the circuitry comprises the necessary hardware (and code, if any is necessary) to perform the function, regardless of whether performance of the function is disabled or not enabled (e.g., by some user-configurable setting, a factory trim, etc.).

Other embodiments may provide a non-transitory computer readable medium and/or storage medium, and/or a non-transitory machine readable medium and/or storage medium, having stored thereon, a machine code and/or a computer program having at least one code section executable by a machine and/or a computer, thereby causing the machine and/or computer to perform the processes as described herein.

Accordingly, the present disclosure may be realized in hardware, software, or a combination of hardware and software. The present disclosure may be realized in a centralized fashion in at least one computing system, or in a distributed fashion where different elements are spread across several interconnected computing systems. Any kind of computing system or other apparatus adapted for carrying out the methods described herein is suited. A typical combination of hardware and software may be a general-purpose computing system with a program or other code that, when being loaded and executed, controls the computing system such that it carries out the methods described herein. Another typical implementation may comprise an application specific integrated circuit or chip.

Various embodiments in accordance with the present disclosure may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. An apparatus for holding and charging a wireless ultrasound probe used in an ultrasound imaging system, the apparatus comprising:
a probe holder configured to securely engage, at least, a probe charger that is configured to engage the wireless ultrasound probe;
wherein the probe holder comprises one or more securing elements configured to secure in place at least the probe charger once engaged with the probe holder;
wherein the probe holder is configured to engage a corresponding part of the ultrasound imaging system; and
wherein the probe holder is configured for being secured in place, once engaged with the ultrasound imaging system, based on a securing mechanism.

2. The apparatus of claim 1, wherein the probe holder comprises or is made from a transparent material.

3. The apparatus of claim 1, wherein the one or more securing elements comprise at least one securing element configured to secure in place, at least in part, both of the probe charger and the wireless ultrasound probe.

4. The apparatus of claim 1, wherein the one or more securing elements comprise at least one element that comprises a hook-like structure extending from an edge of the probe holder and configured for engaging one or both of a side part and a top part of one or both of the probe charger and the wireless ultrasound probe.

5. The apparatus of claim 4, wherein the hook-like structure comprises a double-curve structure comprising a first part for engaging the probe charger and a second part for engaging the wireless ultrasound probe.

6. The apparatus of claim 1, wherein the one or more securing elements comprise at least one element that comprises a protrusion-like structure extending from a top-side of the probe holder when engaged with the corresponding part, the protrusion-like structure configured to engage a bottom-side of the probe charger or a corresponding feature on the bottom-side of the probe charger.

7. The apparatus of claim 1, wherein the probe holder comprise one or more second securing elements configured to secure in place the probe holder, once engaged with the ultrasound imaging system, based on the securing mechanism.

8. The apparatus of claim 7, wherein the securing mechanism comprises magnetic force based mechanism, and wherein the one or more second securing elements comprise at least one structure configured to house a magnet or a steel or iron-based bracket.

9. The apparatus of claim 8, wherein the at least one structure is on a bottom-side of the probe holder.

10. The apparatus of claim 8, wherein the at least one structure is at a location that is opposite a location of at least one corresponding structure on the corresponding part of the ultrasound imaging system, and where the corresponding structure is configured to house a magnet, or to house a steel or iron-based bracket when the at least one structure houses a magnet.

11. The apparatus of claim 1, wherein the corresponding part of the ultrasound imaging system comprises one or more recesses configured to receive the probe holder, and wherein the probe holder is configured to reside or fit with each of the one or more recesses.

12. The apparatus of claim 1, wherein one or both of the probe holder and the corresponding part of the ultrasound imaging system are configured to enable placement of the probe holder at a right-side position or a left-side position with respect to a user of the ultrasound imaging system.

13. An apparatus for holding and charging a wireless ultrasound probe used in an ultrasound imaging system, the apparatus comprising:
a probe charger configured to engage the wireless ultrasound probe; and
a probe holder configured to engage at least the probe charger;
wherein the probe charger is configured to charge the wireless ultrasound probe when engaged therewith;
wherein the probe holder comprises one or more securing elements, and wherein at least one securing element is configured to secure in place at least the probe charger once engaged with the probe holder; and
wherein the probe holder is configured for being secured in place once engaged with the ultrasound imaging system.

14. The apparatus of claim 13, wherein one or both of the probe holder and the probe charger are configured such that when engaged to one another there is space between the probe holder the probe charger to allow for air flow.

15. The apparatus of claim 13, wherein the one or more securing elements comprise at least one element that comprises a hook-like structure extending from an edge of the probe holder and configured for engaging one or both of a side part and a top part of one or both of the probe charger and the wireless ultrasound probe.

16. The apparatus of claim 15, wherein the hook-like structure comprises a double-curve structure comprising a first part for engaging the probe charger and a second part for engaging the wireless ultrasound probe.

17. The apparatus of claim 13, wherein the probe charger comprises one or more structures configured for securing in place, at least in part, the wireless ultrasound probe once engaged therewith.

18. The apparatus of claim 13, wherein the probe charger comprises a port configured to receive a connector for providing power from the ultrasound imaging system via a charging cable.

19. The apparatus of claim 18, wherein the probe holder comprises one or more structures configured to secure in place the charging cable once plugged into the port.

20. An ultrasound imaging system comprising:
a wireless ultrasound probe;
a probe charger configured to engage the wireless ultrasound probe; and
a probe holder configured to engage at least the probe charger;
wherein the probe charger is configured to charge the wireless ultrasound probe when engaged therewith;
wherein the probe holder comprises one or more securing elements, and wherein at least one securing element is configured to secure in place at least the probe charger once engaged with the probe holder; and
wherein the probe holder is configured for being secured in place once engaged with the ultrasound imaging system.
